(19) United States
(12) Patent Application Publication
(10) Pub. No.: US 2004/0062821 A1
(43) Pub. Date: Apr. 1, 2004

(54) Administration of therapeutic compositions for treating fatigue

(76) Inventor: Elena Avram, New York, NY (US)

Correspondence Address: WINSTON & STRAWN, PATENT DEPARTMENT, 1400 L STREET, N.W., WASHINGTON, DC, 20005-3502, US

(21) Appl. No.: 10/261,957

(22) Filed: Oct. 1, 2002

(51) Int. Cl. A61K 31/714; A61K 31/4415; A61K 33/06; A01N 31/04

(52) U.S. Cl. 424/697; 514/168; 514/52; 514/350; 514/725

(57) ABSTRACT

Therapeutic anti-fatigue compositions for administration to a subject including an active combination of a compound of magnesium and sulfur such as magnesium thiosulfate and a mixture of vitamins including A, B₆, B₁₂ and D. The combination is present in the composition in an amount sufficient to treat the symptoms of fatigue in a subject. Methods of using such compositions for treating or managing fatigue are also encompassed by the invention.

### FIELD OF INVENTION

The invention relates to therapeutic compositions for administration to a subject to treat fatigue. The compositions include a compound of magnesium and sulfur and a mixture of vitamins including A, B₆, B₁₂ and D. The components are present in an amount sufficient to treat the symptoms of fatigue in a subject. Methods of using such compositions for treating or managing fatigue are also encompassed by this invention.

### BACKGROUND ART

Many people suffer from fatigue and Chronic Fatigue Syndrome (CFS) each year. Many of these people suffer silently as the symptoms of these conditions are going undiagnosed and untreated.

CFS is an illness characterized by debilitating fatigue and a number of associated symptoms. In September of 1989, the U.S. Center for Disease Control initiated a physician-based Chronic Fatigue Syndrome Surveillance System in four cities to determine the prevalence, incidence, cause and impact of the illness. To date no definitive information has been harvested which applies to CFS as an easily diagnosable malady.

One of the original and remaining diagnostic criteria for CFS is severe, debilitating and unexplainable fatigue, which the patient has experienced and which has occurred for at least the preceding six months. Those suffering from CFS and even regular fatigue not only fail to be properly diagnosed, but even when diagnosed are presented with few choices for effective therapy that can be taken regularly upon the reoccurrence of fatigue symptoms.

At the present time, there is no known reliable composition available to alleviate the severe and debilitating fatigue continuously experienced by persons suffering from CFS and general fatigue. Thus, a need exists for a therapeutic, anti-fatigue composition that is effective in treating CFS and general fatigue, or that can at least abate the symptoms of fatigue.

### SUMMARY OF THE INVENTION

The invention relates to a therapeutic anti-fatigue composition for administration to a subject suffering from fatigue. The anti-fatigue composition typically comprises a compound of magnesium and sulfur and a mixture of vitamins including A, B₆, B₁₂ and D in a therapeutically effective amount sufficient to reduce, inhibit, or avoid fatigue thereof in the patient.

These compositions may be administered in any one of a number of forms, including orally or by injection. Typically, these compositions are administered orally in the form of a tablet or capsule.

The methods for treating or avoiding fatigue in a subject are a further embodiment of this invention. These methods generally comprise administering to a patient in need of such treatment a therapeutically effective amount of one of the therapeutic anti-fatigue compositions disclosed herein.

The invention further encompasses preferred regimes of administering the anti-fatigue compositions so that the compositions effect on fatigue is enhanced and beneficially alleviates or reduces at least some of the symptoms suffered by the subject.

### DETAILED DESCRIPTION OF THE PREFERRED EMBODIMENTS

It has now been discovered that formulations can be prepared that reduce, inhibit, or avoid fatigue or symptoms associated with fatigue, in a subject. It is believed, without being bound by theory, that the surprising and advantageous formulations of the invention operate by controlling the catabolic metabolism rates and/or by up regulation of the immune system, increasing interleukin 2 levels.

Usually, the anti-fatigue composition of the invention includes an active combination that includes a compound of magnesium and sulfur in an amount sufficient to provide an antagonistic catabolic effect and a mixture of vitamins including A, B₆, B₁₂ and D. The vitamin mixture is usually included in an amount sufficient to enhance the effect produced by the magnesium and sulfur compound.

The active combination is preferably present in an amount of about 0.5 to 10% by weight of the composition. The relative amounts of each component in the combination are not critical and preferred amounts for each are given below. Of course, it is recognized that the amounts can vary depending upon the total amount to be administered per day and the number of dosages to be taken. The following amounts are contemplated for a single dosage form that is administered once per day.

Preferably, the compound of magnesium and sulfur is in the form of magnesium thiosulfate and typically in an amount between 0.1 and 10%. It is however preferable that the magnesium thiosulfate be in an amount between 1 and 5%, and most preferable in an amount of 3% by weight.

The vitamin mixture used in the single dose anti-fatigue composition generally includes about 0.5 to 5 mg of vitamin A; about 0.5 to 10 mg of vitamin B₆; about 0.5 to 8 mcg of vitamin B₁₂; and about 5 to 20 mg of vitamin D. More preferably about 1 to 3 mg of vitamin A; about 1 to 5 mg of vitamin B₆; about 1 to 5 mcg of vitamin B₁₂; and about 10 to 15 mg of vitamin D; and most preferably about 1.5 mg of vitamin A; about 3 mg of vitamin B₆; about 2.5 mcg of vitamin B₁₂; and about 12.5 mg of vitamin D, per dosage. Advantageously, the single daily dosage form usually includes about 3000 u to 7000 u of vitamin A and 300 to 700 u of vitamin D.

Generally, the composition also includes a carrier with the anti-fatigue composition with the active combination. The carrier can be any pharmaceutically acceptable diluent or filler as are well known in the art.

The present invention further includes methods for treating fatigue in a subject. Typically, the method involves the administration of the anti-fatigue compositions, described herein, to a subject in need of such treatment, in an amount effective to counteract the effects or symptoms of fatigue in the subject.

The administration of the anti-fatigue composition is usually administered to a human and preferably includes at least an internal/systemic component rather than being merely topical. The composition is most preferably administered orally in the form of a single dosage tablet or capsule that is preferably administered once daily.

Typically, the anti-fatigue composition is administered to the subject daily for at least one to two weeks, more preferably for one to two months and most preferably for four to five months.

A preferred treatment regime consist of administering at least one dosage to the subject daily for at least four months, followed by about two months of no administration to the subject. This is then followed by at least one week of administering to the subject at least one dosage of the anti-fatigue composition daily.

Subjects being administered the anti-fatigue composition often have symptoms, for example, that consist of neurological, neuromuscular symptoms, immunological abnormalities and other symptoms associated with fatigue. Although these symptoms are useful in determining if a subject is suffering from fatigue, the examples above are not all inclusive. It should be noted that symptoms of fatigue may vary drastically and come in many forms.

The magnitude of a therapeutic dose of the composition in the treatment or management of fatigue, could vary with the severity of fatigue symptoms to be treated and the route of administration. The dose, and perhaps the dose frequency, will also vary according to the age, body weight, and response of the individual patient. In general, the total daily dose range, for the conditions are described herein.

Although any suitable route of administration may be employed for providing the subject with an effective dosage of the composition according to the methods of the present invention, preferred routes include, for example, oral and injection, and like forms of administration may be employed. “Injection” typically includes parenteral, intravenous, subcutaneous, and intramuscular routes. Suitable dosage forms include tablets, troches, dispersions, suspensions, solutions, capsules, gel-caps, patches, one-shot injections, needle less injectors, and the like, although oral and injectable dosage forms are preferred. A preferred injection route is intramuscularly.

The anti-fatigue compositions used in the methods of the present invention include the active ingredients described above, and may also contain carriers, excipients and the like, and optionally, other therapeutic ingredients. The active ingredients used in the methods and compositions can be administered individually, as a single composition that contains all the ingredients, or in any combination thereof.

The compositions for use in the methods of the present invention may be prepared in various formulations, such as suspensions, solutions and elixirs; aerosols; or carriers such as starches, sugars, microcrystalline cellulose, diluents, granulating agents, lubricants, binders, disintegrating agents, and the like, in the case of oral solid preparations such as powders, capsules, and tablets, with the oral solid preparations being preferred over the oral liquid preparations. The most preferred oral solid preparations are tablets.

Because of their ease of administration, tablets and capsules represent the most advantageous oral dosage unit form, in which case solid carriers are employed. If desired, tablets may be coated by standard aqueous or non-aqueous techniques.

Anti-fatigue compositions for use in the methods of the present invention may be presented as discrete units such as capsules, cachets, tablets, or aerosol sprays, each containing a predetermined amount of the active ingredient, as a powder, stick, or granules, as creams (e.g., a conditioner), pastes, gels, lotions, syrups, ointments, sponges or cotton applicators, or as a solution or a suspension in an aqueous liquid, a non-aqueous liquid, an oil-in-water emulsion, or a water-in-oil liquid emulsion.

Such compositions may be prepared by any of the available methods, but typically the methods include the step of bringing into association the carrier with the active ingredient. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally, with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

### EXAMPLES

The following examples demonstrate the effectiveness of the administration of the anti-fatigue compositions on subjects suffering from fatigue or CFS.

Patients suffering from symptoms of fatigue were given one anti-fatigue tablet daily for between four and five months. Seventy-four percent (74%) of patients were 26-56 years old, while 13% were younger than 25 and 14% were 51 or more years old. The female to male ratio of the patients treated was 3:1. Eighty-percent (80%) of the patients treated had symptoms of fatigue for at least one year prior to treatment, while 18% of the subjects had symptoms six years or more.

The anti-fatigue tablets contained a combination of vitamins A, B₆, B₁₂ and D and magnesium thiosulfate in specific amounts of 1.5 mg of vitamin A; 3 mg of vitamin B₆; 2.5 mcg of vitamin B₁₂; 12.5 mg of vitamin D, and 3% magnesium thiosulfate by weight.

After being treated for four to five months the patients abstained from taking the anti-fatigue tablets for two months, followed by the re-administration the anti-fatigue tablets daily for one month.

Patients were advised to follow a prudent diet, abstain from all alcoholic beverages and get adequate rest while receiving treatment.

All patients reported feeling a decrease in fatigue symptoms by at least the 7th day of treatment.

The term “about,” as used herein, should generally be understood to refer to both numbers in a range of numerals. Moreover, all numerical ranges herein should be understood to include each whole integer within the range.

Although preferred embodiments of the invention have been described in the foregoing Detailed Description, it will be understood that the invention is not limited to the embodiments disclosed, but is capable of numerous rearrangements and modifications of parts and elements without departing from the spirit of the invention. It will be understood that the chemical details of every design may be slightly different or modified by one of ordinary skill in the art without departing from the teachings of the present invention.

What is claimed is:

1. A therapeutic anti-fatigue composition comprising an active ingredient combination of a compound of magnesium and sulfur in an amount sufficient to provide an antagonistic catabolic effect and a mixture of vitamins including: A, B₆, B₁₂ and D, in an amount sufficient to enhance the effect produced by the magnesium and sulfur compound.

2. The composition according to claim 1, further comprising a carrier wherein the combination is present in a total amount of about 0.5 to 10% by weight of the composition.

3. The composition according to claim 2, in the form of a tablet or capsule for oral administration.

4. The composition according to claim 3, wherein the compound of magnesium and sulfur is magnesium thiosulfate and is present in an amount between 1 and 5% by weight of the composition.

5. The composition according to claim 3, wherein the vitamin mixture in each tablet or capsule includes: about 0.5 to 5 mg of vitamin A; about 0.5-10 mg of vitamin B₆; about 0.5-8 mcg of vitamin B₁₂; and about 5 to 20 mg of vitamin D.

6. The composition according to claim 5, wherein the vitamin mixture includes: 1 to 3 mg of vitamin A; 1 to 5 mg of vitamin B₆; 1 to 4 mcg of vitamin B₁₂; and 10 to 15 mg of vitamin D.

7. A method for treating fatigue in a subject, the method comprising administering to a subject in need of such treatment the composition of claim 1 in an amount effective to counteract the effects or symptoms of fatigue in the subject.

8. The method according to claim 7, wherein the composition is administered to the subject daily for at least one to two months.

9. The method according to claim 7, wherein the therapeutic anti-fatigue composition is in the form of a tablet or capsule for oral administration.

10. The method according to claim 9, wherein the compound of magnesium and sulfur is magnesium thiosulfate and is present in an amount between 1 and 5% by weight of the composition.

11. The method according to claim 10, wherein the vitamin mixture in each tablet or capsule includes: about 0.5 to 5 mg of vitamin A; about 0.5 to 10 mg of vitamin B₆; about 0.5 to 8 mcg of vitamin B₁₂; and about 5 to 20 mg of vitamin D.

12. The method according to claim 11, wherein at least one tablet or capsule is administered orally per day to the subject for at least one week.

13. The method according to claim 12, wherein at least one tablet or capsule is administered orally per day to the subject for at least two months.

14. The method according to claim 11, wherein at least one tablet or capsule is administered orally per day to the subject for at first a period of at least four months, followed by a second period of about two months of no administration of the tablet or capsule to the subject, which is followed by a third period of at least one week wherein at least one tablet or capsule is administered orally per day to the subject.
